(19) United States
(12) Patent Application Publication
(10) Pub. No.: US 2026/0294572 A1
(43) Pub. Date: Oct. 1, 2026

(54) Through-Barrier Connection Systems and Methods

(71) Applicant: Bard Access Systems, Inc., Salt Lake City, UT (US)

(72) Inventors: Zachary Jarrod Traina, Denville, NJ (US); Matthew Lee Kolb, Cottonwood Heights, UT (US); Bradley M. Wilkinson, North Haledon, NJ (US)

(21) Appl. No.: 19/090,218

(22) Filed: Mar. 25, 2025

(51) Int. Cl. A61B 46/13

(52) U.S. Cl. CPC A61B 46/13

(57) ABSTRACT

Through-barrier connection systems and methods establish safe, routine, functional connections between sterile single-use medical devices and non-sterile multiple-use medical devices through procedural barriers. For example, a through-barrier connection system includes a single-use connector of a single-use medical device and a multiple-use connector of a multiple-use medical device or a patch cable. The single-use connector includes a plug wholly concealed in a plug housing for piercing a procedural barrier with its tip section. The multiple-use connector includes a receptacle within a receptacle housing that receives the plug when an insertable portion of the housing receptacle is inserted into a receptacle-receiving cavity of the plug housing. The receptacle includes an internal electrical interface for establishing an electrical connection with an external electrical interface of the plug. The receptacle also includes an extended optical interface wholly concealed in the receptacle for establishing an optical connection with a recessed optical interface of the plug.

### BACKGROUND

Procedural fields are typically established about patients with one or more procedural barriers before medical procedures. For example, sterile fields can be established over or around patients by covering the patients with sterile drapes. Oftentimes, the medical procedures require multiple-use medical devices that cannot be sterilized, which mandate placement of such medical devices under the foregoing drapes; however, sterile single-use medical devices often need to be functionally connected to the multiple-use medical devices. Some existing solutions rely on arbitrarily breaching drapes to make functional connections between sterile single-use medical devices and non-sterile multiple-use medical devices. But arbitrarily breaching such drapes risks contaminating the sterile fields carefully established about the patients. That, and establishing some functional connections such as optical connections between sterile single-use medical devices and non-sterile multiple-use medical devices can be difficult, particularly through arbitrary, self-made breaches in the drapes. What is needed is a through-barrier connection system for establishing safe, routine, functional connections between sterile single-use medical devices and non-sterile multiple-use medical devices through procedural barriers.

Disclosed herein are through-barrier connection systems and methods that address the foregoing.

### SUMMARY

Disclosed herein a through-barrier connection system for establishing a multimodal functional connection between sterile single-use medical devices and non-sterile multiple-use medical devices through procedural barriers. The through-barrier connection system includes, in some embodiments, a single-use connector of a single-use medical device and a multiple-use connector. The single-use connector includes a plug housing including a receptacle-receiving cavity. The single-use connector also includes a plug extending from a closed end portion of the plug housing toward an opposite, open end portion of the plug housing but wholly concealed in the plug housing. The plug includes a tip section with an angled leading edge configured to pierce a procedural barrier. The plug also includes an external electrical interface for establishing an electrical connection and a recessed optical interface recessed into the plug for establishing an optical connection. The multiple-use connector includes a receptacle housing including an insertable portion that inserts into the receptacle-receiving cavity of the plug housing. The multiple-use connector also includes a receptacle within the receptacle housing that receives the plug. The receptacle includes an internal electrical interface for establishing the electrical connection with the external electrical interface of the plug of the single-use connector. The receptacle also includes an extended optical interface extending from a closed end portion of the receptacle toward an opposite, open end portion of the receptacle but wholly concealed in the receptacle. The extended optical interface establishes the optical connection with the recessed optical interface of the plug of the single-use connector.

In some embodiments, the insertable portion of the receptacle housing and the receptacle-receiving cavity of the plug housing have transverse cross-sectional geometries that complement each other and enforce rotational alignment of the receptacle housing and the plug housing along a common longitudinal axis when inserting the insertable portion of the receptacle housing into the receptacle-receiving cavity of the plug housing.

In some embodiments, an engineering tolerance between the insertable portion of the receptacle housing and the receptacle-receiving cavity of the plug housing allows the insertable portion of the receptacle housing to be inserted into the receptacle-receiving cavity of the plug housing along with the procedural barrier around the insertable portion of the receptacle housing.

In some embodiments, the single-use connector further includes a latch handle, and the multiple-use connector further includes a latch pad for latching the single-use connector onto the multiple-use connector after inserting the insertable portion of the receptacle housing into the receptacle-receiving cavity of the plug housing.

In some embodiments, the external electrical interface of the plug is a transversely curved metal strip over a molded plug body that terminates with the tip section.

In some embodiments, the metal strip includes a pair of wings extending from an end portion of the metal strip immediately adjacent the tip section of the molded plug. The pair of wings wrap at least partially around the plug body for securing the metal strip to the plug body.

In some embodiments, the internal electrical interface of the receptacle is a spring-loaded metal strip extending from a printed circuit board (“PCB”) into the receptacle between the extended optical interface and the open end portion of the receptacle.

In some embodiments, the extended optical interface includes an alignment sleeve disposed in an extension of a molded receptacle body extending from a closed end portion of the receptacle body coextensive with the closed end portion of the receptacle.

In some embodiments, the extended optical interface includes at least a portion of an optical-fiber-holding ferrule spring-loaded in the alignment sleeve. The ferrule extends into the alignment sleeve through a ferrule opening in the closed end portion of the receptacle.

In some embodiments, the recessed optical interface includes an optical-fiber-holding ferrule spring-loaded in a bore of the plug. The bore of the plug has an inner diameter commensurate with an outer diameter of the extension of the molded receptacle body for inserting the extension of the molded receptacle body into the bore of the plug to establish the optical connection.

In some embodiments, the multiple-use connector is a distal multimodal connector of a multiple-use medical device.

In some embodiments, the multiple-use connector is a distal multimodal connector of a ‘Y’-shaped patch cable. The patch cable bifurcates in a proximal portion thereof to provide a pair of proximal connectors including an electrical connector and an optical connector for connecting to a multiple-use medical device.

In some embodiments, the single-use medical device is a central venous access device (“CVAD”) and the multiple-use medical device being a tip location confirmation system (“TLCS”).

Also disclosed herein is a single-use medical device including, in some embodiments, a single-use connector for establishing a multimodal functional connection with a multiple-use connector of a multiple-use medical device through a procedural barrier. The single-use connector includes a plug housing including a receptacle-receiving cavity. The single-use connector also includes a plug extending from a closed end portion of the plug housing toward an opposite, open end portion of the plug housing but wholly concealed in the plug housing. The plug includes a tip section with an angled leading edge configured to pierce the procedural barrier. The plug also includes an external electrical interface for establishing an electrical connection with the multiple-use connector and a recessed optical interface recessed into the plug for establishing an optical connection with the multiple-use connector.

In some embodiments, the single-use medical device further includes a protective cap disposed over the open end portion of the plug housing, in the receptacle-receiving cavity over the plug, or over the open end portion of the plug housing with an extension of the protective cap disposed over the plug for protecting the plug.

In some embodiments, the external electrical interface of the plug is a transversely curved metal strip over a molded plug body that terminates with the tip section.

In some embodiments, the metal strip includes a pair of wings extending from an end portion of the metal strip immediately adjacent the tip section of the molded plug. The pair of wings wrap at least partially around the plug body for securing the metal strip to the plug body.

In some embodiments, the single-use connector further includes a latch handle for latching the single-use connector onto the multiple-use connector after establishing the multimodal functional connection with the multiple-use connector.

In some embodiments, the recessed optical interface includes an optical-fiber-holding ferrule spring-loaded in a bore of the plug.

In some embodiments, the single-use medical device is a CVAD.

Also disclosed herein is a patch cable including, in some embodiment, a multiple-use connector for establishing a multimodal functional connection with a single-use connector of a single-use medical device through a procedural barrier. The multiple-use connector includes a receptacle housing including an insertable portion that inserts into the single-use connector. The multiple-use connector also includes a receptacle within the receptacle housing. The receptacle includes an internal electrical interface for establishing an electrical connection with the single-use connector. The receptacle also includes an extended optical interface extending from a closed end portion of the receptacle toward an opposite, open end portion of the receptacle but wholly concealed in the receptacle. The extended optical interface establishes an optical connection with the single-use connector.

In some embodiments, the multiple-use connector further includes a latch pad for latching the single-use connector onto the multiple-use connector after establishing the multimodal functional connection with the single-use connector.

In some embodiments, the internal electrical interface of the receptacle is a spring-loaded metal strip extending from a PCB into the receptacle between the extended optical interface and the open end portion of the receptacle.

In some embodiments, the extended optical interface includes an alignment sleeve disposed in an extension of a molded receptacle body extending from a closed end portion of the receptacle body coextensive with the closed end portion of the receptacle.

In some embodiments, the extended optical interface includes at least a portion of an optical-fiber-holding ferrule spring-loaded in the alignment sleeve. The ferrule extends into the alignment sleeve through a ferrule opening in the closed end portion of the receptacle.

In some embodiments, the multiple-use connector is a distal multimodal connector of a ‘Y’-shaped patch cable. The patch cable bifurcates in a proximal portion thereof to provide a pair of proximal connectors including an electrical connector and an optical connector for connecting to a multiple-use medical device.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates an isometric view of a single-use connector and a multiple-use connector of a through-barrier connection system in accordance with some embodiments.

FIG. 2 illustrates another isometric view of the single-use connector and the multiple-use connector of the through-barrier connection system in accordance with some embodiments.

FIG. 3 illustrates a longitudinal cross-section of the multiple-use connector in accordance with some embodiments.

FIG. 4 illustrates a longitudinal cross-section of the single-use connector in accordance with some embodiments.

FIG. 5 illustrates an isometric view of the single-use connector with a protective cap in accordance with some embodiments.

FIG. 6 illustrates a longitudinal cross-section of the single-use connector with the protective cap in accordance with some embodiments.

FIG. 7 illustrates a longitudinal cross-section of each of the single-use connector and the multiple-use connector prior to establishing a through-barrier connection through a procedural drape in accordance with some embodiments.

FIG. 8 illustrates a longitudinal cross-section of each of the single-use connector and the multiple-use connector after establishing the through-barrier connection through the procedural drape in accordance with some embodiments.

FIG. 9 illustrates an end-on view of a connecting end of the multiple-use connector in accordance with some embodiments.

FIG. 10 illustrates an end-on view of a connecting end of the single-use connector in accordance with some embodiments.

FIG. 11 illustrates electrical and optical interfaces of the single-use and multiple-use connectors in accordance with some embodiments.

FIG. 12 illustrates the through-barrier connection system with a patch cable thereof in use during a medical procedure in accordance with some embodiments.

FIG. 13 illustrates the through-barrier connection system with a tip location confirmation system in use during a medical procedure in accordance with some embodiments.

FIG. 14 illustrates a method of using the through-barrier connection system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

“Single use,” is used to indicate, for example, a single-use medical device, a single-use connector, or the like is intended for use with a single patient before discarding the single-use medical device, the single-use connector, or the like. However, it should be understood that “single use” need not indicate that the single-use medical device, the single-use connector, or the like is used a single time, for a single function, with a particular duration, or some combination thereof on the foregoing single patient. “Multiple use” contrasts with “single use” in that “multiple use” is used to indicate, for example, a multiple-use medical device, a multiple-use connector, or the like is intended for use with multiple patients before any need for refurbishing or discarding the multiple-use medical device, the multiple-use connector, or the like.

Unless defined otherwise, all technical and scientific terms used herein have the same meaning as commonly understood by those of ordinary skill in the art.

### Through-Barrier Connection Systems

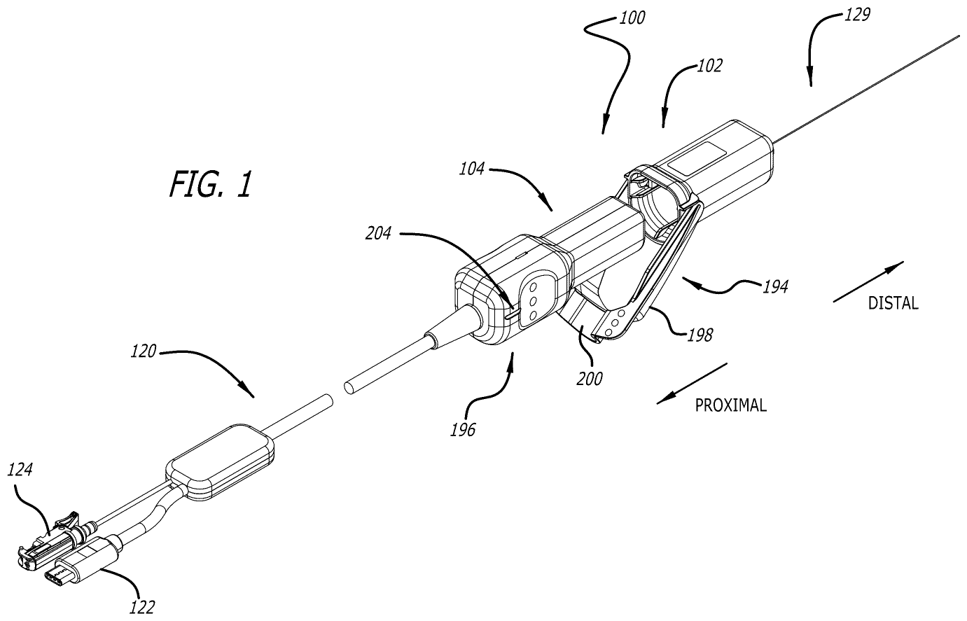

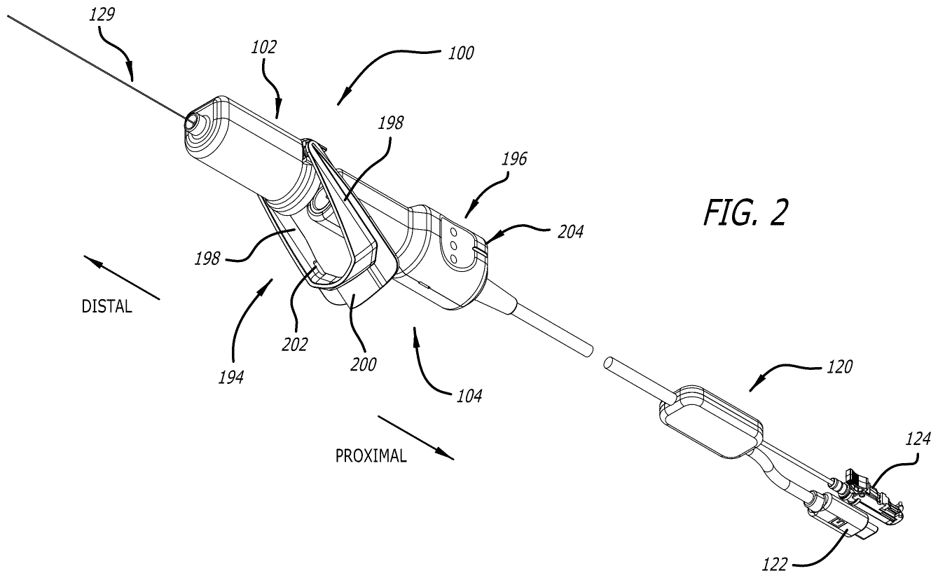

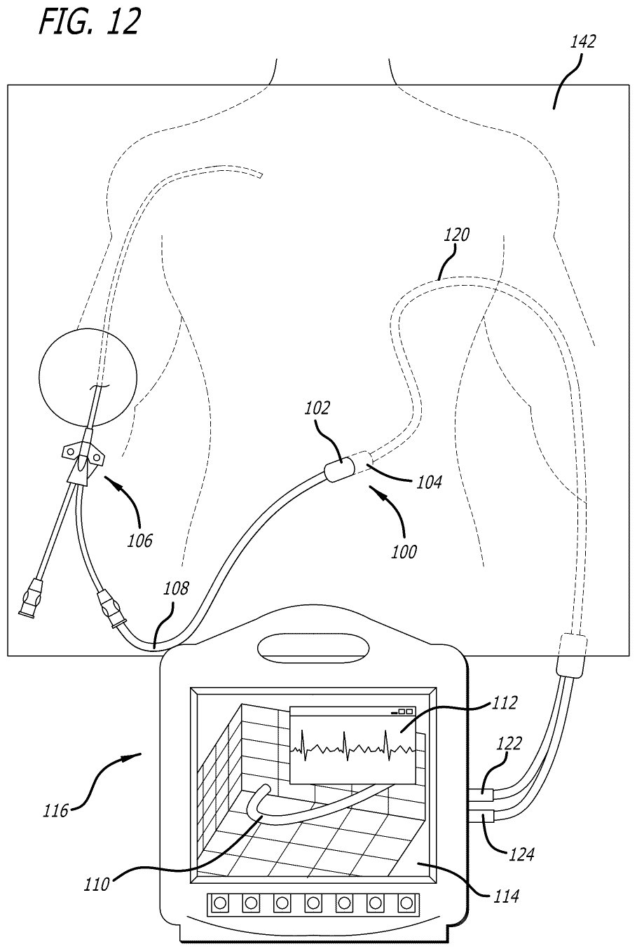

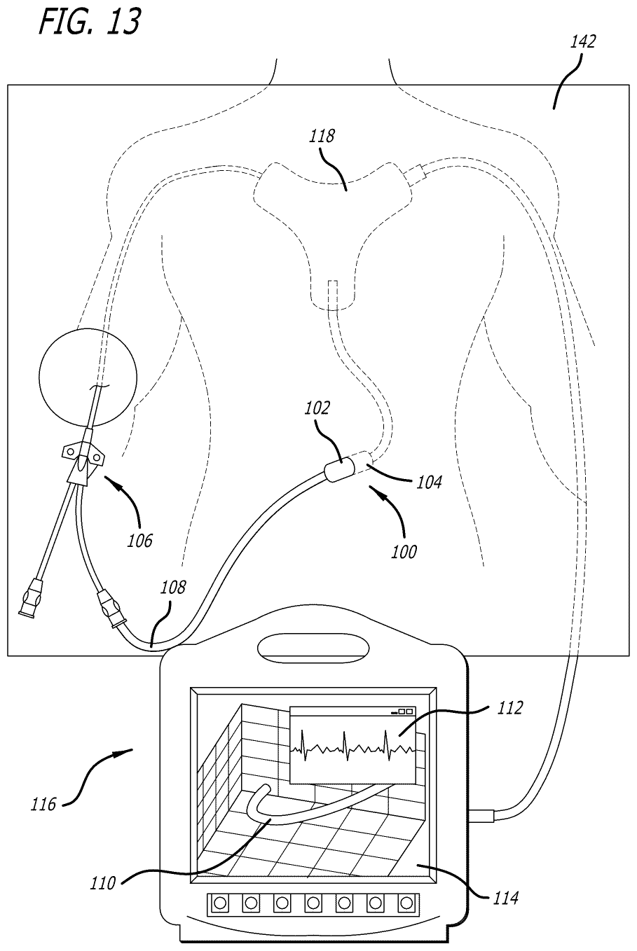

FIGS. 1 and 2 illustrate different views of a single-use connector 102 and a multiple-use connector 104 of a through-barrier connection system 100 in accordance with some embodiments. FIGS. 12 and 13 illustrates the through-barrier connection system 100 in use during a medical procedure in accordance with some embodiments.

As shown, the through-barrier connection system 100 can include the single-use connector 102 as a proximal connector of a single-use medical device including, but not limited to, a central venous access device (“CVAD”) such as a peripherally inserted central catheter (“PICC”) 106 or a medical device associated therewith such as a probe, particularly, a shape-sensing electrocardiogram (“ECG”) stylet 108 as conveyed in FIGS. 12 and 13 by way of a three-dimensional (“3-D”) sensed shape 110 of the stylet 108 along with a corresponding electrocardiogram 116 on a display screen 114 of a console 116. The through-barrier connection system 100 can also include the multiple-use connector 104 as a distal connector of a multiple-use medical device including, but not limited to, a tip location confirmation system (“TLCS”) 118 for placing the foregoing CVAD or confirming its location after placement. Alternatively, the multiple-use connector 104 is a distal connector of a patch cable 120 between the single-use medical device and the console 116 or a multiple-use medical device such as the TLCS 118.

When the multiple-use connector 104 is the distal connector of the patch cable 120, a proximal portion of the patch cable 120 can be split for a ‘Y’-shaped proximal portion of the patch cable 120. As shown, such a patch cable 120 bifurcates in the proximal portion thereof to provide a pair of proximal connectors including an electrical, data, or electrical-and-data connector 122 and an optical connector 124 for connecting the single-use medical device and the console 116 or a multiple-use medical device such as the TLCS 118. Notably, the electrical, data, or electrical-and-data connector 122 is operably connected to the spring-loaded metal strip 178 through the printed circuit board (“PCB”) 180 set forth below by one or more metal wires extending through the patch cable 120. Like the optical-fiber-holding ferrule 186 of the extended optical interface 182 set forth below, the optical connector 124 includes an optical-fiber-holding ferrule that holds the optical fiber 132 extending through the patch cable 120.

Such a through-barrier connection system 100 is configured for establishing multimodal functional connections including, but not limited to, optical, electrical, and data connections between sterile single-use medical devices and non-sterile multiple-use medical devices or other non-sterile capital equipment including the console 116 through procedural barriers such as disposable sterile drapes. Accordingly, the single-use connector 102 can be considered a proximal multimodal connector of a single-use multimodal medical device, and the multiple-use connector 104 can be considered a distal multimodal connector of either a multiple-use multimodal medical device or a multimodal patch cable between the single-use multimodal medical device and a multiple-use multimodal medical device or the console 116. However, it should be understood that while the through-barrier connection system 100 is configured for establishing multimodal functional connections, the through-barrier connection system 100 can be modified for establishing fewer connections than the foregoing optical, electrical, and data connections such as any two functional connections selected from the foregoing connections for a bimodal functional connection. Further, it should be understood that the through-barrier connection system 100 can be further modified for establishing a unimodal functional connection including an optical, electrical, or data connection. In such modified through-barrier connection systems, superfluous connection componentry can be removed provided a piercing means such as the plug 138 for piercing the procedural barriers remains.

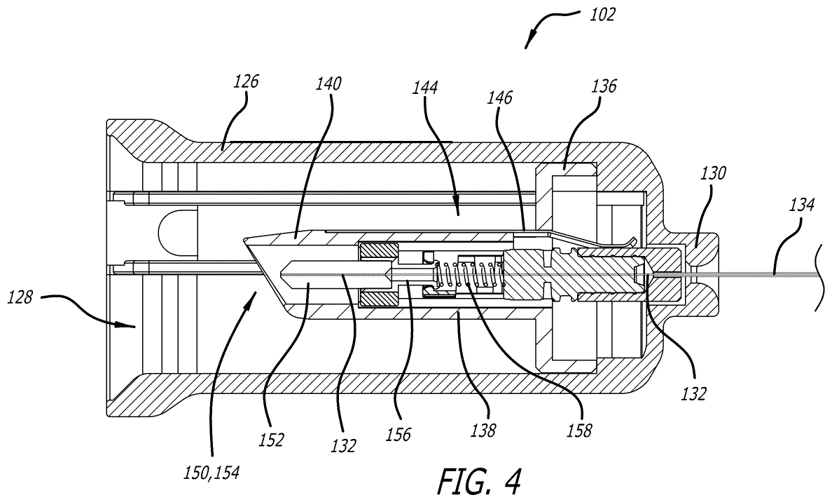

FIGS. 4-6 and 10 illustrate different views of the single-use connector 102 in accordance with some embodiments.

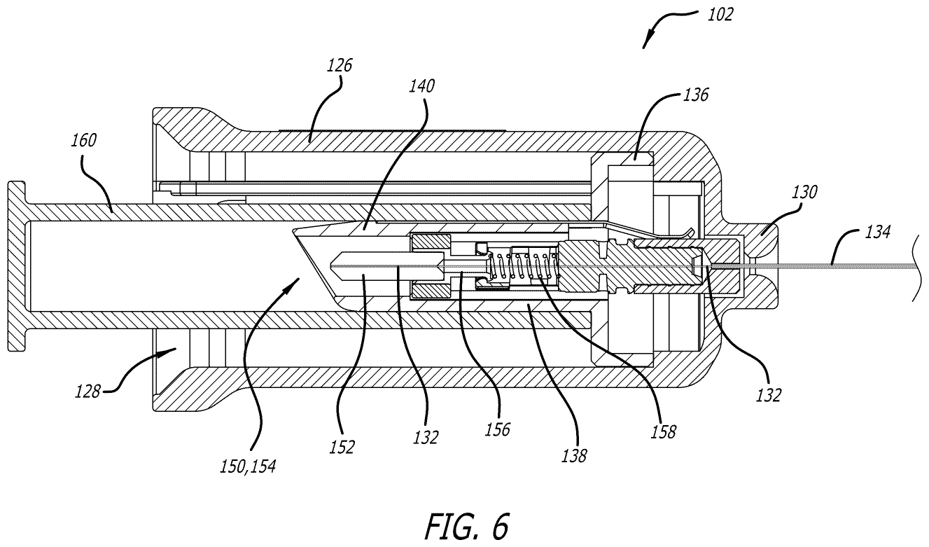

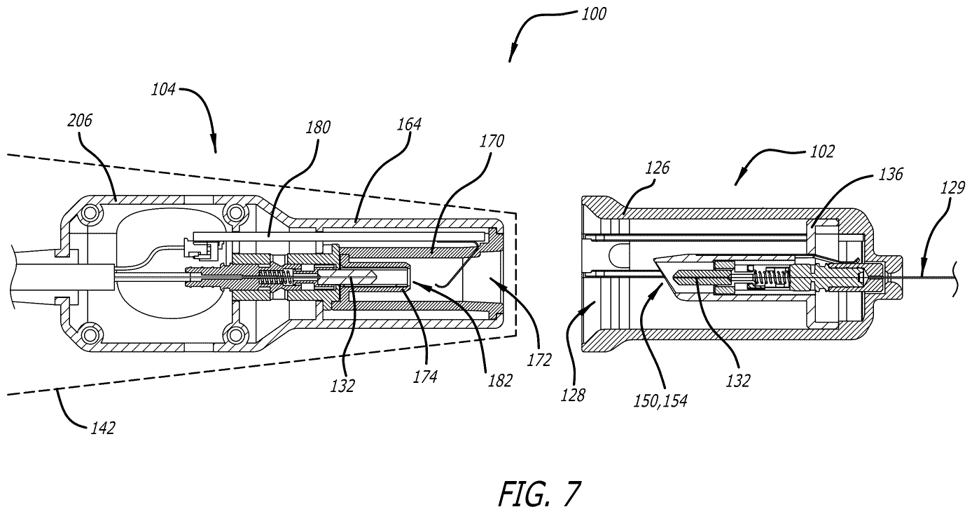

As shown, the single-use connector 102 can include a molded plug shroud or plug housing 126 including a receptacle-receiving cavity 128. The plug housing 126 can be molded such that the receptacle-receiving cavity 128 thereof has a transverse cross-sectional geometry that complements the insertable portion 164 of the receptacle housing 162 to enforce rotational alignment of the receptacle housing 162 and the plug housing 126 along a common longitudinal axis (discerned by an optical-fiber assembly 129 shown in FIG. 7) when inserting the insertable portion 164 of the receptacle housing 162 into the receptacle-receiving cavity 128 of the plug housing 126. Further, the plug housing 126 can be molded with channels, passageways, or the like to accommodate electrical leads and optical fibers from the single-use medical device. For example, the plug housing 126 shown in FIGS. 4 and 6 has a boss 130 through which a multicore optical fiber 132 disposed in a casing 134 (e.g., a nitinol tube), extends from the single-use medical device into the recessed optical interface 150 of the plug 138.

The single-use connector 102 can also include a molded plug body 136 disposed in the plug housing 126 defining a plug 138 that extends from a closed end portion of the plug housing 126 toward an opposite, open end portion of the plug housing 126. While the plug 138 extends toward the open end portion of the plug housing 126, it is wholly concealed in the plug housing 126, as shown, thereby preventing incidental contact or stick injuries of a clinician, a patient, or the like. Such a plug 138 can include a tip section 140 with an angled leading edge configured to pierce a procedural barrier 142, notably, when the procedural barrier 142 is pushed into the open end portion of the plug housing 126 by the insertable portion 164 of the receptacle housing 162. Advantageously, the tip section 140 with its angled leading edge is configured to pierce the procedural barrier 142 without coring the procedural barrier 142, thereby preventing any detached portion of the procedural barrier 142 from interfering with any of the functional connections set forth herein.

FIGS. 4, 6, and 11 illustrate an external electrical interface 144 of the single-use connector 102 in accordance with some embodiments.

The plug 138 can include the external electrical interface 144 for establishing an electrical connection, a data connection, or both such as that consistent with an ECG probe of the single-use medical device. As shown, the external electrical interface 144 of the plug 138 can be a transversely curved metal strip 146 over the plug body 136 or plug 138 thereof that terminates with the tip section 140 of the plug 138. Notably, the metal strip 146 can include a pair of wings 148 extending from an end portion of the metal strip 146 immediately adjacent the tip section 140 of the plug 138. Such a pair of wings 148 can wrap at least partially around the plug body 136 or plug 138 thereof for securing the metal strip 146 thereto.

FIGS. 4, 6, and 11 also illustrate a recessed optical interface 150 of the single-use connector 102 in accordance with some embodiments.

The plug 138 can include the recessed optical interface 150 recessed into the plug 138 for establishing an optical connection such as that consistent with a shape-sensing probe of the single-use medical device. As shown, the recessed optical interface 150 of the plug 138 can include an optical-fiber-holding ferrule 152 spring-loaded and wholly concealed in a bore 154 of the plug body 136 or plug 138 thereof. Notably, a portion of the ferrule 152 is disposed in a ferrule carrier 156 inside the bore 154, which ferrule carrier 156 is operably connected to a compression spring 158 that provides the spring force for spring loading the ferrule 152 in the bore 154 of the plug body 136 or plug 138 thereof. The bore 154 has an inner diameter commensurate with an outer diameter of the extension 174 of the receptacle body 170 for inserting the extension 174 into the bore 154 of the plug body 136 or plug 138 thereof to establish the optical connection. Advantageously, such an optical connection is recessed in accordance with both the single-use connector 102 and the multiple-use connector 104 to prevent incidental contact of optical components with the procedural barrier 142, a clinician, or the like, thereby maintaining fidelity of the optical connection.

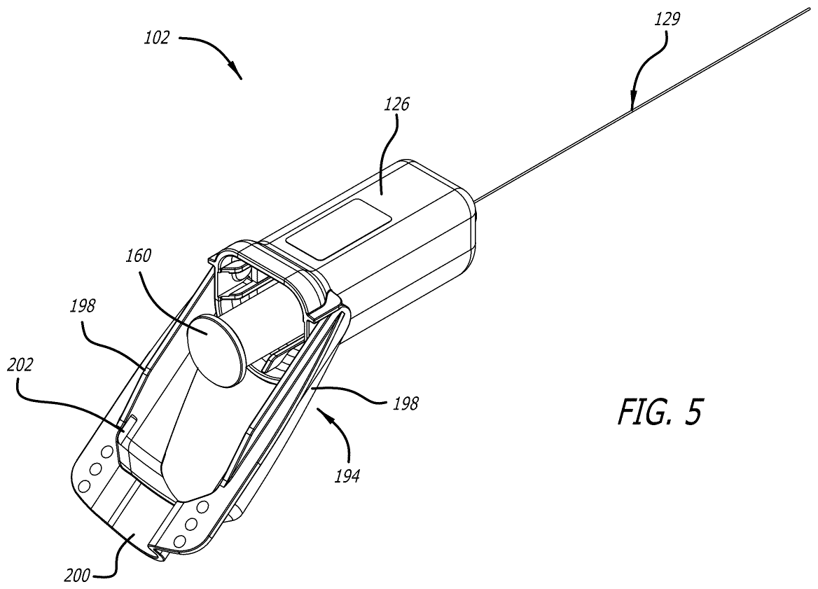

FIGS. 5 and 6 illustrate different views of the single-use connector 102 with a protective cap 160 in accordance with some embodiments.

As shown, the single-use medical device 102 can also include the protective cap disposed over the open end portion of the plug housing 126, in the receptacle-receiving cavity 128 over the plug 138, as shown, or over the open end portion of the plug housing 126 with a central extension of the protective cap 160 akin to that shown disposed over the plug 138. Regardless of its configuration, the protective cap 160 is for protecting the plug 138 prior to establishing any functional connections with the single-use connector 102.

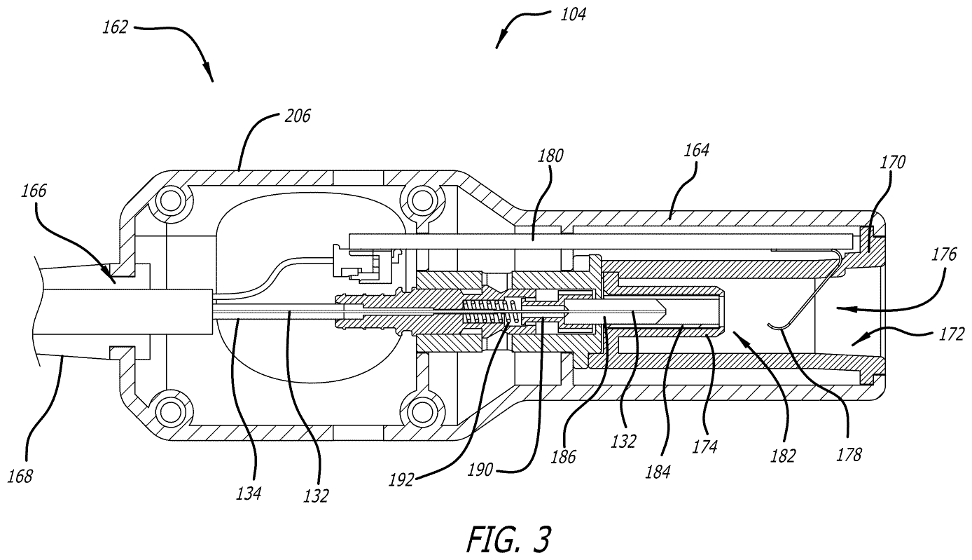

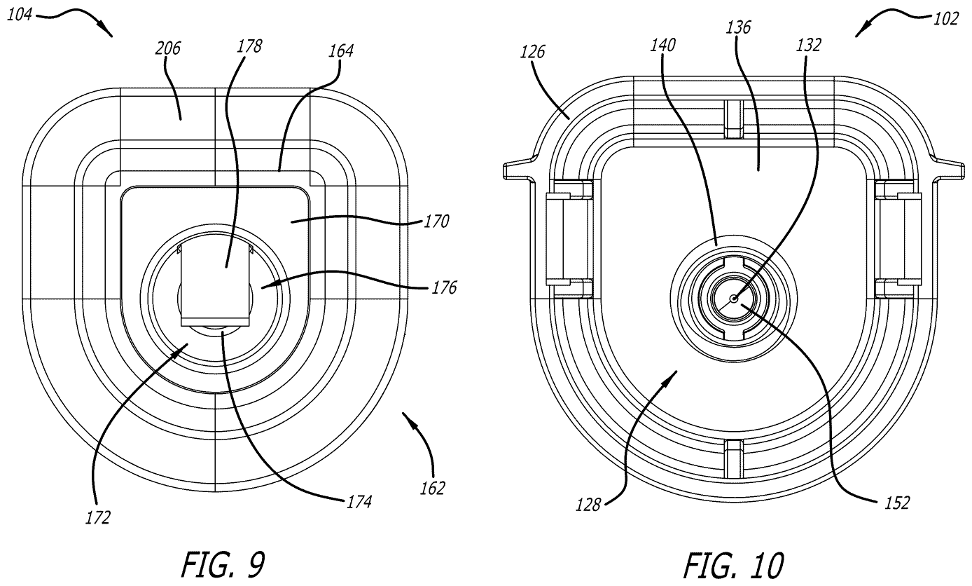

FIGS. 3 and 9 illustrates different views of the multiple-use connector 104 in accordance with some embodiments.

As shown, the multiple-use connector 104 can include a molded receptacle housing 162 including an insertable portion 164 that inserts into the receptacle-receiving cavity 128 of the plug housing 126 through the open end portion thereof. The receptacle housing 162 can be molded such that the insertable portion 164 thereof has a transverse cross-sectional geometry that complements the receptacle-receiving cavity 128 of the plug housing 126 to enforce rotational alignment of the receptacle housing 162 and the plug housing 126 along a common longitudinal axis (discerned by the optical-fiber assembly 129 shown in FIG. 7) when inserting the insertable portion 164 of the receptacle housing 162 into the receptacle-receiving cavity 128 of the plug housing 126. Further, the receptacle housing 162 can be molded with channels, passageways, or the like to accommodate electrical leads and optical fibers from the multiple-use medical device or patch cable 120. For example, the receptacle housing 162 shown in FIG. 3 has an aperture 166 and a strain-relief boot 168 disposed therein through which the multicore optical fiber 132 disposed in the casing 134 (e.g., a polymer tube), extends from the multiple-use medical device or patch cable 120 into the extended optical interface 182 of the receptacle 172.

The multiple-use connector 104 can also include a molded receptacle body 170 disposed in the receptacle housing 162 defining a receptacle 172 that extends from an internal portion of the receptacle housing 162 toward an open end portion of the receptacle housing 162. The receptacle body 170 or receptacle 172 thereof can include a closed end portion and an opposite, open end portion that is coextensive with the open end portion of the receptacle housing 162 that receives the plug 138 of the single-use connector 102. Further, the receptacle body 170 can include an extension 174 extending from the closed end portion of the receptacle body 170 into the receptacle 172 for the extended optical interface 182.

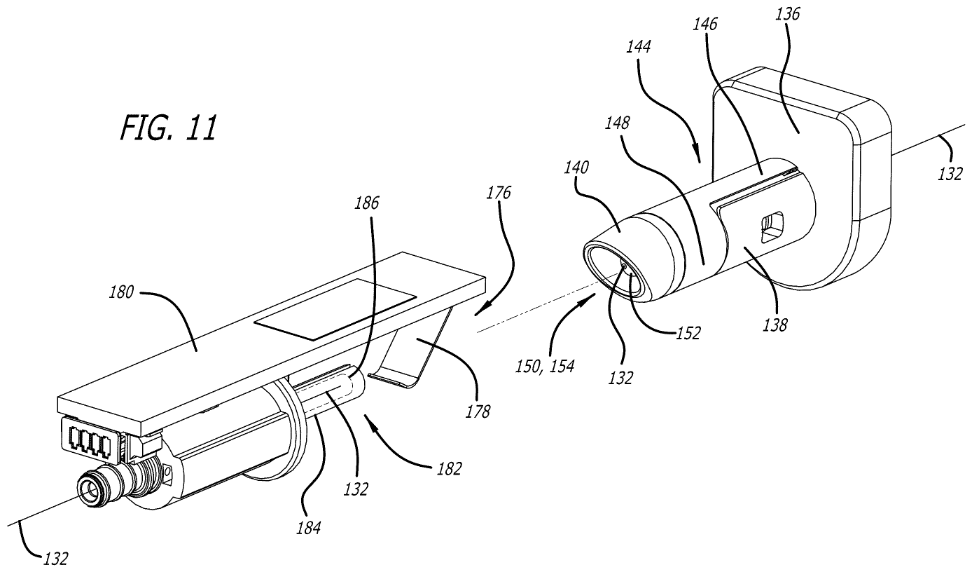

FIG. 11 illustrates an internal electrical interface 176 of the multiple-use connector 104 in accordance with some embodiments.

The receptacle 172 can include the internal electrical interface 176 for establishing the electrical connection, the data connection, or both with the external electrical interface 144 of the plug 138 of the single-use connector 102. As shown, the internal electrical interface 176 of the receptacle 172 can be a spring-loaded metal strip 178 akin to a cantilever spring extending from a PCB 180 into the receptacle 172 through a longitudinal slot in the receptacle body 170 (discerned by the metal strip 178 over a wall of the receptacle body 170 in FIG. 3). The metal strip 178 is located between the extended optical interface 182 and the open end portion of the receptacle 172 such that the metal strip 178 is compressed in the foregoing longitudinal slot between the plug 138 of the single-use connector 102 and the PCB 180 before the extension 174 in the receptacle 172 inserts into the bore 154 of the plug 138.

FIG. 11 also illustrates an extended optical interface 182 of the multiple-use connector 104 in accordance with some embodiments.

The receptacle 172 can include the extended optical interface 182 for establishing the optical connection with the recessed optical interface 150 of the plug 138 of the single-use connector 102. As shown, the extended optical interface 182 can extend from the closed end portion of the receptacle 172 toward the open end portion of the receptacle 172, but the extended optical interface 182 remains wholly concealed in the receptacle housing 162 or receptacle 172 thereof, thereby providing another recessed optical interface of the through-barrier connection system 100 for preventing incidental contact of optical components with the procedural barrier 142, a clinician, or the like as set forth above. Such an extended optical interface 182 can include an alignment sleeve 184 disposed in the extension 174 extending from the closed end portion of the receptacle body 170 that is coextensive with the closed end portion of the receptacle 172. Further, at least a portion of an optical-fiber-holding ferrule 186 can be spring-loaded in the alignment sleeve 184 through a ferrule opening in the closed end portion of the receptacle housing 162 or receptacle 172 thereof. Notably, a portion of the ferrule 186 is disposed in a ferrule carrier 190 outside the alignment sleeve 184, which ferrule carrier 190 is operably connected to a compression spring 192 that provides the spring force for spring loading the ferrule 186 in the alignment sleeve 184 disposed in the extension 174 of the receptacle body 170 or receptacle 172 thereof.

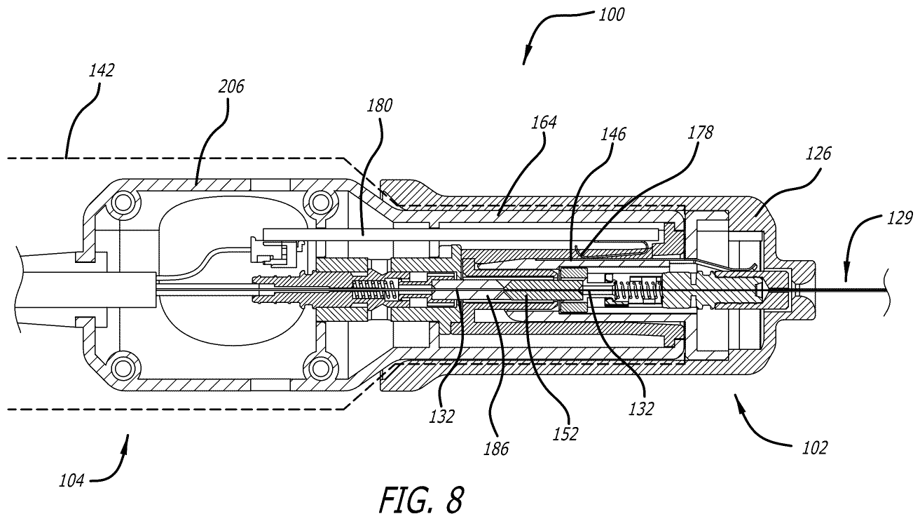

FIG. 7 illustrates a longitudinal cross-section of each of the single-use connector 102 and the multiple-use connector 104 prior to establishing a through-barrier connection through the procedural barrier 142 in accordance with some embodiments. FIG. 8 illustrates a longitudinal cross-section of each of the single-use connector 102 and the multiple-use connector 104 after establishing the through-barrier connection through the procedural barrier 142 in accordance with some embodiments. Further, FIGS. 9 and 10 illustrate end-on views of the single-use connector 102 and the multiple-use connector 104 from their connecting ends in accordance with some embodiments.

As shown, the insertable portion 164 of the receptacle housing 162 and the receptacle-receiving cavity 128 of the plug housing 126 have transverse cross-sectional geometries that complement each other and enforce rotational alignment of the receptacle housing 162 and the plug housing 126 along a common longitudinal axis (discerned by the optical-fiber assembly 129 shown in FIG. 7) when inserting the insertable portion 164 of the receptacle housing 162 into the receptacle-receiving cavity 128 of the plug housing 126. Notably, when the insertable portion 164 of the receptacle housing 162 is inserted into the receptacle-receiving cavity 128 of the plug housing 126, the receptacle housing 162 and the plug housing 126 form a prismatic joint that constrains any motion of the receptacle housing 162 and the plug housing 126 to sliding along their common longitudinal axis without rotation. However, such sliding is further limited by the latch handle 194 of the single-use connector 102 and the complementary latch pad 196 of the multiple-use connector 104 set forth below for latching the single-use connector 102 onto the multiple-use connector 104.

Notwithstanding the foregoing, instead of the insertable portion 164 of the receptacle housing 162 and the receptacle-receiving cavity 128 of the plug housing 126 having the transverse cross-sectional geometries that enforce rotational alignment of the receptacle housing 162 and the plug housing 126 along their common longitudinal axis, the insertable portion 164 of the receptacle housing 162 and the receptacle-receiving cavity 128 of the plug housing 126 can include a key and keyway to enforce the rotational alignment of the receptacle housing 162 and the plug housing 126 along their common longitudinal axis when inserting the insertable portion 164 of the receptacle housing 162 into the receptacle-receiving cavity 128 of the plug housing 126. Such a key and keyway allow transverse cross-sectional geometries between the insertable portion 164 of the receptacle housing 162 and the receptacle-receiving cavity 128 of the plug housing 126 that would otherwise allow discrete or continuous rotation between the insertable portion 164 of the receptacle housing 162 and the receptacle-receiving cavity 128 of the plug housing 126, for example, transverse cross-sectional geometries that are circular. Either one of the insertable portion 164 of the receptacle housing 162 or the receptacle-receiving cavity 128 of the plug housing 126 can include the key while the other one of the insertable portion 164 of the receptacle housing 162 or the receptacle-receiving cavity 128 of the plug housing 126 includes the keyway.

No matter how rotational alignment of the receptacle housing 162 and the plug housing 126 is enforced along their common longitudinal axis, when the insertable portion 164 of the receptacle housing 162 is inserted into the receptacle-receiving cavity 128 of the plug housing 126, the receptacle housing 162 and the plug housing 126 form, as set forth above, a motion-constraining prismatic joint. Constraining any motion of the receptacle housing 162 and the plug housing 126 to sliding along their common longitudinal axis without rotation ensures the internal electrical interface 176 of the receptacle 172 of the multiple-use connector 104 remains in contact with the external electrical interface 144 of the plug 138 of the single-use connector 102 for establishing the electrical connection, the data connection, or both therebetween. However, it should be understood that the external electrical interface 144 of the plug 138 can fully surround the plug 138 like a jacket in some embodiments, thereby obviating enforcement of any particular rotational alignment of the receptacle housing 162 and the plug housing 126 along their common longitudinal axis. Should rotation need to be restricted between the receptacle housing 162 and the plug housing 126 in such embodiments, for example, to prevent torsional strain on a distal portion of the single-use medical device, the latch handle 194 of the single-use connector 102 and the complementary latch pad 196 of the multiple-use connector 104 can be further configured to restrict rotation between the receptacle housing 162 and the plug housing 126 as set forth below.

An engineering tolerance between the insertable portion 164 of the receptacle housing 162 and the receptacle-receiving cavity 128 of the plug housing 126 allows the insertable portion 164 of the receptacle housing 162 to be inserted into the receptacle-receiving cavity 128 of the plug housing 126 along with the procedural barrier 142 around the insertable portion 164 of the receptacle housing 162. Accordingly, while inserting the insertable portion 164 of the receptacle housing 162 into the receptacle-receiving cavity 128 of the plug housing 126, the procedural barrier 142 is drawn over the insertable portion 164 of the receptacle housing 162 with sufficient tautness for the tip section 140 of the plug 138 with its angled leading edge to pierce the procedural barrier 142 as the plug 138 of the single-use connector 102 enters the receptacle 172 of the multiple-use connector 104.

As best shown in FIGS. 1 and 2, the through-barrier connection system 100 can also include a securement mechanism for securing the single-use connector 102 and the multiple-use connector 104 together upon their connection to each other. Such a securement mechanism can include, but is not limited to, a latch handle 194 on the single-use connector 102 and a latch pad 196 on the multiple-use connector 104 for latching the single-use connector 102 onto the multiple-use connector 104 after inserting the insertable portion 164 of the receptacle housing 162 into the receptacle-receiving cavity 128 of the plug housing 126. However, it should be understood that the inverse of the foregoing is also possible with the latch handle 194 on the multiple-use connector 104 and the latch pad 196 on the single-use connector 102. Continuing with the illustrated embodiment, the latch handle 194 can approximate a ‘U’ shape with arms 198 of the latch handle 194 connected together by a base or bridge 200 of the latch handle 194. Such a latch handle 194 can be rotatably coupled to the closed end portion of the plug housing 126 by tips of the arms 198 of the latch handle 194. The bridge 200 connecting the arms 198 of the latch handle 194 together can include a pair of inward-facing protrusions 202, and the latch pad 196 of the multiple use connector can include a complementary pair of outward-facing indentations 204 for receiving the pair of protrusions 202 when the bridge 200 of the latch handle 194 is rotated over a handle portion 206 of the receptacle housing 162 including the latch pad 196, notably, after inserting the insertable portion 164 of the receptacle housing 162 into the receptacle-receiving cavity 128 of the plug housing 126. Advantageously, such a securement mechanism can maintain a compressive force on the compression springs 158 and 192 associated with the optical-fiber holding ferrules 152 and 186 of the single-use and multiple-used connector 102 and 104 subsequent to inserting the insertable portion 164 of the receptacle housing 162 into the receptacle-receiving cavity 128 of the plug housing 126 and establishing the optical connection. Further, a flat portion of the bridge 200 of the latch handle 194 having the pair of protrusions 202 flushly engages a flat portion of the latch pad 196 having the pair of indentations 204, the flat portions of the latch handle 194 and the latch pad 196 thereby restricting rotation between the receptacle housing 162 and the plug housing 126 when mated.

### Methods

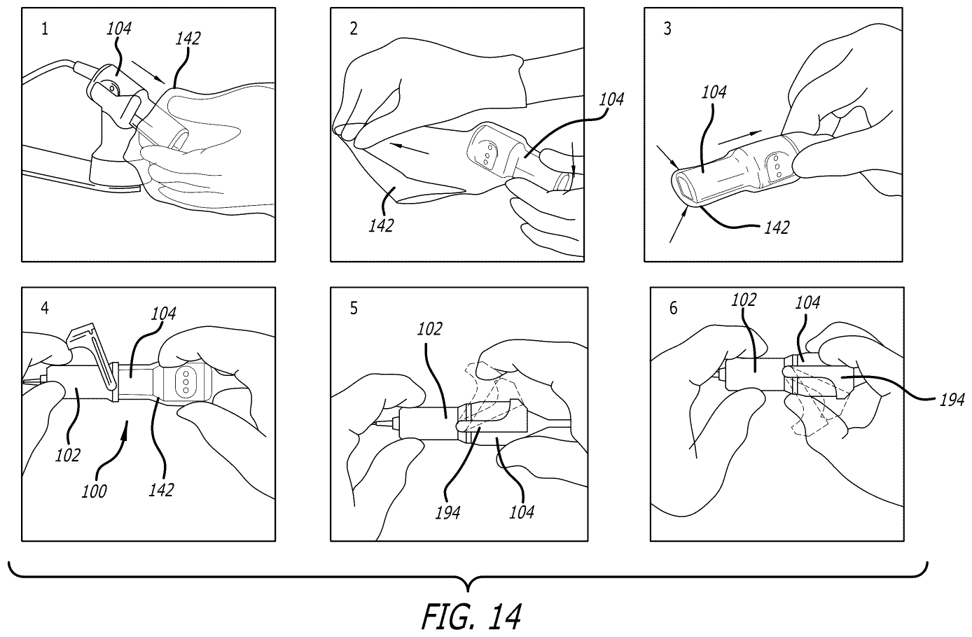

FIG. 14 illustrates at least a portion of a method of using the through-barrier connection system 100 in accordance with some embodiments.

As shown, the method can include aseptically covering or draping a non-sterile multiple-use connector 104 with the procedural barrier 142 (e.g., a sterile drape) and, while the multiple-use connector 104 remains covered or draped in the procedural barrier 142, inserting the insertable portion 164 of the receptacle housing 162 of the multiple-use connector 104 into the receptacle-receiving cavity 128 of the plug housing 126 of a sterile single-use connector 102. Again, while inserting the insertable portion 164 of the receptacle housing 162 into the receptacle-receiving cavity 128 of the plug housing 126, the procedural barrier 142 is drawn over the insertable portion 164 of the receptacle housing 162 with sufficient tautness for the tip section 140 of the plug 138 with its angled leading edge to pierce the procedural barrier 142 as the plug 138 of the single-use connector 102 enters the receptacle 172 of the multiple-use connector 104, thereby establishing a sterile, multimodal functional connection through the procedural barrier 142. Further, the method can include latching or otherwise securing the single-use connector 102 onto the multiple-use connector 104 after inserting the insertable portion 164 of the receptacle housing 162 into the receptacle-receiving cavity 128 of the plug housing 126. Such latching can include rotating the latch handle 194 of the plug housing 126 over the handle portion 206 of the receptacle housing 162 until the pair of indentations 204 of the latch pad 196 receive the pair of protrusions 202 of the latch handle 194. Subsequent to placing, for example, the PICC 106 set forth above using the shape-sensing ECG stylet 108, the method can include unlatching or otherwise unsecuring the single-use connector 102 from the multiple-use connector 104. Such unlatching can include rotating the latch handle 194 of the plug housing 126 away from the handle portion 206 of the receptacle housing 162 to free the pair of protrusions 202 of the latch handle 194 from the pair of indentations 204 of the latch pad 196. Further, the method can include withdrawing the insertable portion 164 of the receptacle housing 162 of the multiple-use connector 104 from the receptacle-receiving cavity 128 of the plug housing 126 of the single-use connector 102 and, optionally, discarding the single-use medical device including the single-use connector 102.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A through-barrier connection system for establishing a multimodal functional connection between sterile single-use medical devices and non-sterile multiple-use medical devices through procedural barriers, comprising:
a single-use connector of a single-use medical device, the single-use connector including:
a plug housing including a receptacle-receiving cavity; and
a plug extending from a closed end portion of the plug housing toward an opposite, open end portion of the plug housing but wholly concealed in the plug housing, the plug including:
a tip section with an angled leading edge configured to pierce a procedural barrier;
an external electrical interface for establishing an electrical connection; and
a recessed optical interface recessed into the plug for establishing an optical connection; and
a multiple-use connector including:
a receptacle housing including an insertable portion that inserts into the receptacle-receiving cavity of the plug housing; and
a receptacle within the receptacle housing that receives the plug, the receptacle including:
an internal electrical interface for establishing the electrical connection with the external electrical interface of the plug of the single-use connector; and
an extended optical interface extending from a closed end portion of the receptacle toward an opposite, open end portion of the receptacle but wholly concealed in the receptacle, the extended optical interface for establishing the optical connection with the recessed optical interface of the plug of the single-use connector.

2. The through-barrier connection system of claim 1, the insertable portion of the receptacle housing and the receptacle-receiving cavity of the plug housing having transverse cross-sectional geometries that complement each other and enforce rotational alignment of the receptacle housing and the plug housing along a common longitudinal axis when inserting the insertable portion of the receptacle housing into the receptacle-receiving cavity of the plug housing.

3. The through-barrier connection system of claim 1, an engineering tolerance between the insertable portion of the receptacle housing and the receptacle-receiving cavity of the plug housing allowing the insertable portion of the receptacle housing to be inserted into the receptacle-receiving cavity of the plug housing along with the procedural barrier around the insertable portion of the receptacle housing.

4. The through-barrier connection system of claim 1, the single-use connector further including a latch handle and the multiple-use connector further including a latch pad for latching the single-use connector onto the multiple-use connector after inserting the insertable portion of the receptacle housing into the receptacle-receiving cavity of the plug housing.

5. The through-barrier connection system of claim 1, the external electrical interface of the plug being a transversely curved metal strip over a molded plug body that terminates with the tip section.

6. The through-barrier connection system of claim 5, the metal strip including a pair of wings extending from an end portion of the metal strip immediately adjacent the tip section of the molded plug, the pair of wings wrapped at least partially around the plug body for securing the metal strip to the plug body.

7. The through-barrier connection system of claim 1, the internal electrical interface of the receptacle being a spring-loaded metal strip extending from a printed circuit board (“PCB”) into the receptacle between the extended optical interface and the open end portion of the receptacle.

8. The through-barrier connection system of claim 1, the extended optical interface including an alignment sleeve disposed in an extension of a molded receptacle body extending from a closed end portion of the receptacle body coextensive with the closed end portion of the receptacle.

9. The through-barrier connection system of claim 8, the extended optical interface including at least a portion of an optical-fiber-holding ferrule spring-loaded in the alignment sleeve, the ferrule extending into the alignment sleeve through a ferrule opening in the closed end portion of the receptacle.

10. The through-barrier connection system of claim 8, the recessed optical interface including an optical-fiber-holding ferrule spring-loaded in a bore of the plug, the bore of the plug having an inner diameter commensurate with an outer diameter of the extension of the molded receptacle body for inserting the extension of the molded receptacle body into the bore of the plug to establish the optical connection.

11. The through-barrier connection system of claim 1, the multiple-use connector being a distal multimodal connector of a multiple-use medical device.

12. The through-barrier connection system of claim 1, the multiple-use connector being a distal multimodal connector of a ‘Y’-shaped patch cable, the patch cable bifurcating in a proximal portion thereof to provide a pair of proximal connectors including an electrical connector and an optical connector for connecting to a multiple-use medical device.

13. The through-barrier connection system of claim 12, the single-use medical device being a central venous access device (“CVAD”) and the multiple-use medical device being a tip location confirmation system (“TLCS”).

14. A single-use medical device, comprising:
a single-use connector for establishing a multimodal functional connection with a multiple-use connector of a multiple-use medical device through a procedural barrier, the single-use connector including:
a plug housing including a receptacle-receiving cavity; and
a plug extending from a closed end portion of the plug housing toward an opposite, open end portion of the plug housing but wholly concealed in the plug housing, the plug including:
a tip section with an angled leading edge configured to pierce the procedural barrier;
an external electrical interface for establishing an electrical connection with the multiple-use connector; and
a recessed optical interface recessed into the plug for establishing an optical connection with the multiple-use connector.

15. The single-use medical device of claim 14, further including a protective cap disposed over the open end portion of the plug housing, in the receptacle-receiving cavity over the plug, or over the open end portion of the plug housing with an extension of the protective cap disposed over the plug for protecting the plug.

16. The single-use medical device of claim 14, the external electrical interface of the plug being a transversely curved metal strip over a molded plug body that terminates with the tip section.

17. The single-use medical device of claim 16, the metal strip including a pair of wings extending from an end portion of the metal strip immediately adjacent the tip section of the molded plug, the pair of wings wrapped at least partially around the plug body for securing the metal strip to the plug body.

18. The single-use medical device of claim 14, the single-use connector further including a latch handle for latching the single-use connector onto the multiple-use connector after establishing the multimodal functional connection with the multiple-use connector.

19. The single-use medical device of claim 14, the recessed optical interface including an optical-fiber-holding ferrule spring-loaded in a bore of the plug.

20. The single-use medical device of claim 14, the single-use medical device being a central venous access device (“CVAD”).

21. A patch cable, comprising:
a multiple-use connector for establishing a multimodal functional connection with a single-use connector of a single-use medical device through a procedural barrier, the multiple-use connector including:
a receptacle housing including an insertable portion that inserts into the single-use connector; and
a receptacle within the receptacle housing, the receptacle including:
an internal electrical interface for establishing an electrical connection with the single-use connector; and
an extended optical interface extending from a closed end portion of the receptacle toward an opposite, open end portion of the receptacle but wholly concealed in the receptacle, the extended optical interface for establishing an optical connection with the single-use connector.

22. The patch cable of claim 21, the multiple-use connector further including a latch pad for latching the single-use connector onto the multiple-use connector after establishing the multimodal functional connection with the single-use connector.

23. The patch cable of claim 21, the internal electrical interface of the receptacle being a spring-loaded metal strip extending from a printed circuit board (“PCB”) into the receptacle between the extended optical interface and the open end portion of the receptacle.

24. The patch cable of claim 21, the extended optical interface including an alignment sleeve disposed in an extension of a molded receptacle body extending from a closed end portion of the receptacle body coextensive with the closed end portion of the receptacle.

25. The patch cable of claim 24, the extended optical interface including at least a portion of an optical-fiber-holding ferrule spring-loaded in the alignment sleeve, the ferrule extending into the alignment sleeve through a ferrule opening in the closed end portion of the receptacle.

26. The patch cable of claim 21, the multiple-use connector being a distal multimodal connector of a ‘Y’-shaped patch cable, the patch cable bifurcating in a proximal portion thereof to provide a pair of proximal connectors including an electrical connector and an optical connector for connecting to a multiple-use medical device.
